(19) United States
(12) Patent Application Publication
(10) Pub. No.: US 2008/0039693 A1
(43) Pub. Date: Feb. 14, 2008

(54) ENDOSCOPE TIP UNIT AND ENDOSCOPE WITH SCANNING OPTICAL FIBER

(75) Inventor: Satoshi Karasawa, Saitama (JP)

Correspondence Address: GREENBLUM & BERNSTEIN, P.L.C., 1950 ROLAND CLARKE PLACE, RESTON, VA, 20191, US

(73) Assignees: University of Washington, Seattle, WA (US); PENTAX Corporation, Tokyo (JP)

(21) Appl. No.: 11/464,319

(22) Filed: Aug. 14, 2006

(51) Int. Cl. A61B 1/06; A61B 1/04

(52) U.S. Cl. 600/175; 600/110; 600/136; 600/129; 600/182

(57) ABSTRACT

An endoscope has an insertion portion and a tip unit detachably attached to a distal end of the insertion portion. The insertion portion is configured to be inserted into an object, and has a signal cable and a first optical fiber extending therethrough. The tip unit has a second optical fiber optically connected to the first optical fiber, and an actuator electrically connected to the signal cable. The actuator vibrates the second optical fiber so as to scan light irradiated from the second optical fiber over an observed area.

### BACKGROUND OF THE INVENTION

1. Field of the Invention

The present invention relates to an endoscope with an optical fiber which transmits light emitted from a light source to the distal end of the endoscope. In particular, it relates to an endoscope with a scanning optical fiber that vibrates in resonance to scan a beam over a surface to be observed.

2. Description of the Related Art

In an endoscope with a scanning optical fiber, a single mode optical fiber is attached to a piezoelectric 2-D actuator so that the distal end of the fiber becomes a cantilever beam. The piezoelectric actuator 2-dimensionally vibrates the cantilevered distal end at a resonant frequency while modulating or amplifying amplitudes of the vibration, so that the tip portion of the optical fiber is driven in a spiral pattern. A plurality of photodetectors, which are provided around the distal end of the optical fiber, detect light reflected from a region of tissue, and a sequence of signals are transmitted from the photodetectors to a processor connected to the endoscope. The spiral scanning is repeatedly performed by a frame rate, so that a full-color video-image is displayed on a monitor connected to the processor.

### SUMMARY OF THE INVENTION

An object of the present invention is to provide an endoscope that can perform various diagnostic functions with reduced cost and effort. An endoscope according to the present invention may be a flexible or rigid endoscope, such as a video-scope with an image sensor or a fiber-scope, which is equipped with a tubular insertion portion, and an endoscope tip unit. The tubular insertion portion is inserted into an object to be observed, such as an organ of a body or an engine cylinder. A signal cable and an optical fiber pass through the insertion portion. A plurality of endoscope tip units may be provided, and an endoscope tip unit suitable for an observation situation is selectively attached to the insertion portion of an endoscope. Further, a disposable endoscope tip unit can be attached to the distal end of the endoscope, in which case the endoscope tip unit is replaced for each diagnosis.

According to one aspect of the present invention, there is provided an endoscope including an insertion portion and a tip unit removably attached to a distal end of the insertion portion. The insertion portion is configured to be inserted into an object, and has a signal cable and a first optical fiber extending therethrough. The tip unit includes a second optical fiber optically connected to the first optical fiber, and an actuator electrically connected to the signal cable. The actuator vibrates the second optical fiber so as to scan light irradiated from the second optical fiber over an observed area.

The tip unit may include a flexible board that electrically connects the actuator with the signal cable. The flexible board may be arranged laterally with respect to the second optical fiber.

The endoscope may include a pin electrically connected to the signal cable and provided at the distal end of the insertion portion; and a receptacle provided at a connecting portion of the tip unit and configured to receive the pin. The tip unit may include a ferrule that connects the second optical fiber with the first optical fiber. A sleeve that accommodates the ferrule may be provided.

The second optical fiber may be a single mode optical fiber. The actuator nay be a piezoelectric actuator. The actuator may vibrate a tip portion of the second optical fiber in spiral patterns. The tip unit may include at least one optical lens located at a tip portion of the tip unit. The tip unit may include at least one photodetector electrically connected to the signal cable and configured to convert light reflected or emitted by the observed area to image-pixel signals. The tip unit may include at least one filter that blocks part of light reflected or emitted by the observed area. The tip unit may support the second optical fiber along a longitudinal axis of the endoscope tip unit. The tip unit may be configured to detect auto-fluorescence emitted by the observed area. The tip unit may be configured for side viewing of the observed area.

According to another aspect of the present invention, there is provided an endoscope tip unit including a connector that has an electrical connection portion and an optical connection portion; an optical fiber that is optically connected to the optical connection portion; an actuator that is electrically connected to the electrical connection portion, the actuator vibrating the optical fiber so as to scan light irradiated from the optical fiber over an observed area; and a tubular housing that accommodates the optical fiber. The tip unit is configured to be removably attached to a distal end of an endoscope insertion portion.

The optical fiber may be disposed along a longitudinal axis of the endoscope tip unit. The connector may include a sleeve that is coaxial to the housing. The optical connection portion may be coaxial to the housing. The optical connection portion may include a ferrule that optically connects the optical fiber with the optical connection portion. The connector may include a sleeve that coaxially accommodates the ferrule.

The endoscope tip unit may include a flexible board that electrically connects the actuator with the electrical connection portion. The actuator may include a tubular piezoelectric actuator, the optical fiber being passed through the actuator along a longitudinal axis of the endoscope tip unit. The actuator may vibrate a tip portion of the optical fiber in spiral patterns. The endoscope tip unit may further include at least one optical lens located at a tip portion of the endoscope tip unit.

The endoscope tip unit may further include at least one photodetector electrically connected to the electrical connection portion and configured to convert light reflected or emitted by the observed area to image-pixel signals. The endoscope tip unit may further include at least one filter that blocks a part of light reflected or emitted by the observed area. The electric connection portion may have a receptacle. The optical fiber may be a single mode optical fiber. The tip unit may be configured to detect auto-fluorescence emitted by the observed area. The tip unit may be configured for side viewing of the observed area. The electrical connection portion may be configured to connect to a signal cable of the endoscope insertion portion. The optical connection portion may be configured to connect to an optical fiber of the endoscope insertion portion.

According to another aspect of the present invention, there is provided an endoscope including an operation portion that is operated to manipulate a distal end of the endoscope, and a tubular insertion portion configured to be inserted into an object and removably attached to the operation portion. A first signal cable and a first optical fiber extend from a proximal end of the endoscope through the operation portion. The insertion portion includes a second optical fiber optically connected to the first optical fiber, a second signal cable electrically connected to the first signal cable, and an actuator electrically connected to the second signal cable and vibrating a tip portion of the second optical fiber so as to scan light irradiated from the second optical fiber over an observed area. The insertion portion may include an instrument channel.

### BRIEF DESCRIPTION OF THE DRAWINGS

The present invention is further described in the detail description which follows, in reference to the noted plurality of drawings, by way of non-limiting examples of preferred embodiments of the present invention, in which like characters represent like elements throughout the several views of the drawings, and wherein:

FIG. 1 is a block diagram of an endoscope system according to an embodiment of the invention;

FIG. 2 is a perspective view showing a tip unit of the endoscope;

FIG. 3 is a sectional perspective view showing an inner construction of the tip unit;

FIG. 4 is a schematic cross-section view of a connector of the tip unit;

FIG. 5A is a perspective view showing a second type of tip unit;

FIG. 5B is a perspective view showing a third type of tip unit; and

FIG. 6 is a sectional perspective view showing an endoscope according to another embodiment of the invention.

### DETAILED DESCRIPTION

The present invention will be described below with reference to the embodiments shown in the drawings.

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

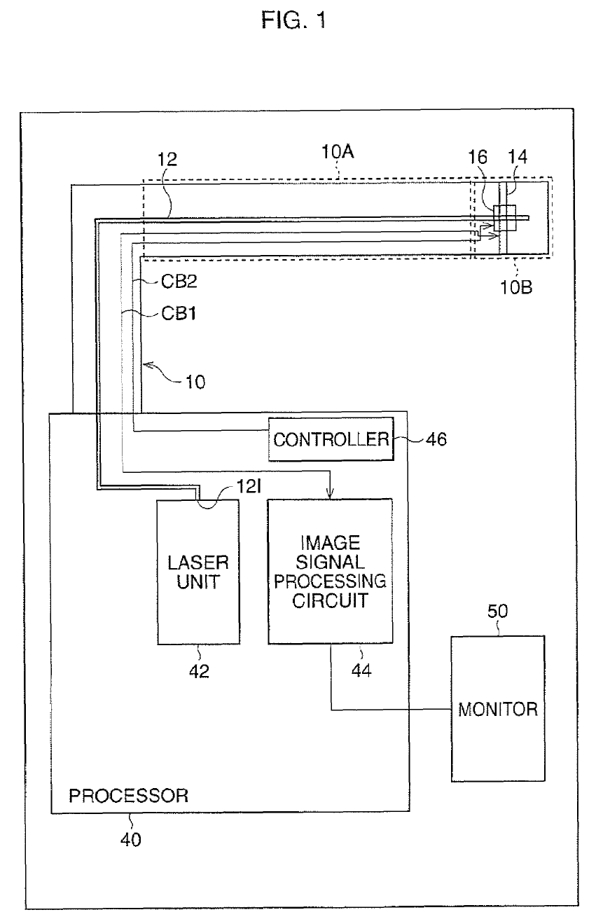

FIG. 1 is a block diagram of an endoscope system according to a first embodiment.

The endoscope system has an endoscope 10, a processor 40, and a monitor 50. The endoscope 10 is detachably connected to the processor 40. A tubular and flexible insertion portion 10A of the endoscope 10, which is connected to an operation portion, is inserted into a body to diagnose or operate on a diseased portion. A rigid or stiff tip unit 10B is detachably or removably attached to the distal end of the insertion portion 10A. The monitor 50 is connected to the processor 40.

A laser unit 42 is provided in the processor 40 and emits a laser bean. The irradiated light enters an incident surface 12I of an optical fiber 12, which may be a single-mode optical fiber. The optical fiber 12 extends through the endoscope 10 and guides or directs the light from the proximal end to the distal end of the endoscope 10. Light passing through the optical fiber 12 exits from the tip portion of the endoscope 10, so that an observed area or portion is illuminated.

Light reflected off, or emitted by, the observed portion enters a plurality of photodiodes 14, which are provided at the distal end of the endoscope 10. Image-pixel signals are successively sent from the photodiodes 14 to an image signal processing circuit 44 in a processor 40 via a signal cable CB1. In the image signal processing circuit 44, the image-pixel signals are subjected to various processes to generate image signals. The generated image signals are fed to the monitor 50, so that an object image is displayed on the monitor 50.

A controller 46 outputs control signals to the distal end of the endoscope 10 via a signal cable CB2 to control an actuator 16. Any suitable actuator may be provided, such as a piezoelectric actuator. A timing controller outputs clock pulse signals to a photodiode driver so as to synchronize a read-timing of the image-pixel signals with the driving of the actuator 16.

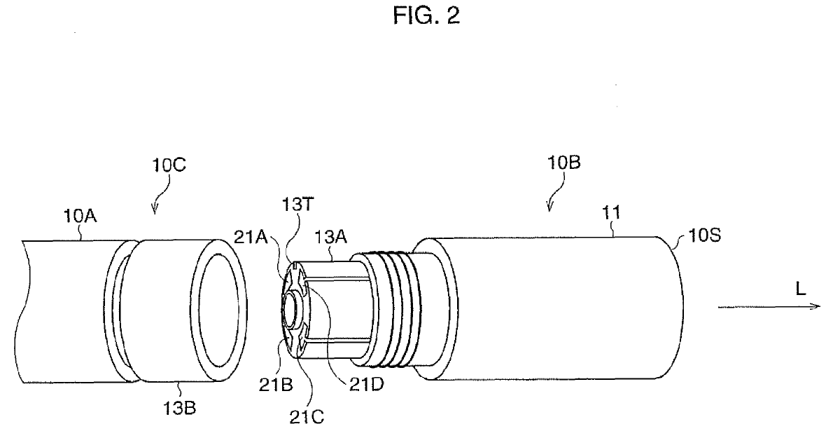

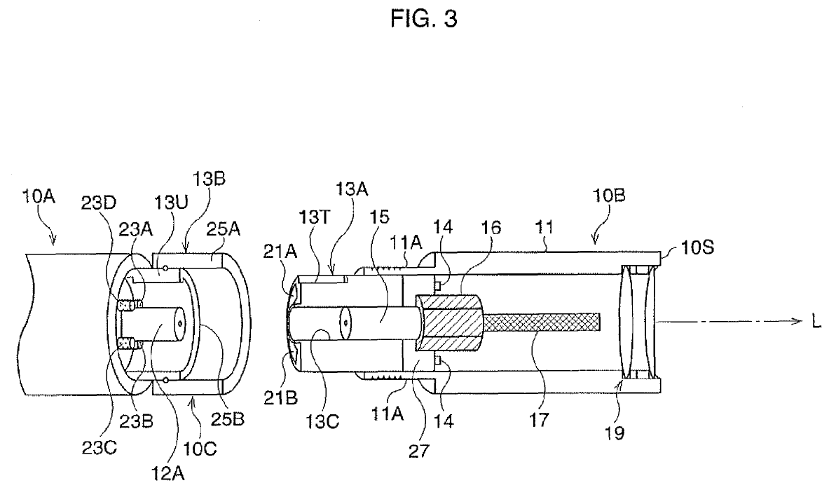

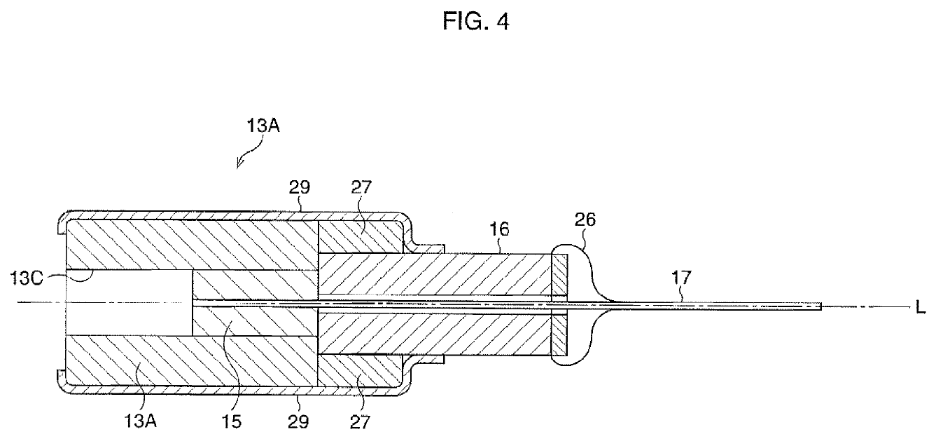

FIG. 2 is a view showing the tip unit 10B of the endoscope 10 shown in FIG. 1. FIG. 3 is a view showing an inner construction of the tip unit 10B. FIG. 4 is a schematic cross-section view of a connector of the tip unit 10B.

As shown in FIG. 2, the tubular tip unit 10B, which is detachably or removably attached to the distal end 10C of the insertion portion 10A, has a housing 11 and a connector 13A. The tip unit 10B is a forward observation type tip unit that acquires an image of a forward direction, which the tip surface 10S of the tip unit 10B faces. The tip surface 10S of the tip unit 10B faces the direction along the endoscope's axis or longitudinal direction L. The sleeve-shaped connector 13A is fixed to one end portion of the tubular and cylindrical housing 11, and detachably fits in a rigid connector 13B, which is fixed to the distal end 10C of the insertion portion 10A.

As shown in FIG. 3, an optical system 19 including two lenses is arranged at the tip portion of the tip unit 10B, and an optical fiber 17 (hereinafter, called “vibrating optical fiber”) is accommodated in the housing 11. The optical system 19 deflects light passing through the vibrating optical fiber 17 toward the tissue. An IR cut filter may be provided in the housing 11. Of course, any suitable optical system may be provided.

The piezoelectric actuator 16 is attached to the connector 13A by a fixing member 27, and the vibrating optical fiber 17 passes through and extends from the piezoelectric actuator 16 toward the optical system 19. A ferrule 15 is inserted into a hole 13C of the sleeve-like connector 13A, which coaxially extends along the axis L. The length of the ferrule 15 is shorter than that of the hole 13C.

The connector 13B, fixed to the distal end 10C of the insertion portion 10A, is a double housing constructions which includes an outer housing 25A and an inner housing 25B. A cylindrical protrusion 11A of the housing 11, which extends toward the connector 13B, is received in the outer housing 25A, whereas the connector 13A of the tip unit 10B is received in the inner housing 25B. An alignment groove 13T of the connector 13A extends along the axis L, and a guide protrusion or extension 13U is formed on the inner surface of the inner housing 25B so as to engage with the groove 13T. Alternatively, a guide protrusion may be provided on the connector 13A for engagement with an alignment groove provided on the inner housing 25B. The tip unit 10B may be fixed to the connector 13B in any suitable manner, such as by a screw. The tip unit 10B may be attached to or detached from the connector 13B by turning the screw. Further, a suitable sealing device, such as an O-ring, may be positioned between the connector 13A and the connector 13B.

In the inner housing 25B, the end portion 12A of the optical fiber 12 coaxially projects along the axis L. The end portion 12A is inserted in the coaxial hole 13C formed in the connector 13A, and connects with the ferrule 15. In this manner the vibrating optical fiber 17 optically connects with the optical fiber 12.

Four metallic pins 23A, 23B, 23C, and 23D connect with the signal cables CB1 and CB2 shown in FIG. 1, and extend toward the connector 13A in the inner housing 25B. The pins 23A, 23B, 23C, and 23D are disposed laterally to the optical fiber end portion 12A at regular intervals. Four sockets or receptacles 21A to 21D are provided around the hole 13C of the connector 13A so as to be disposed opposite to the four pins 23A to 23D. When the tip unit 10B is attached to the distal end 10C of the insertion portion 10A, the four pins 23A to 23D fit into the four receptacles 21A to 21D shown in FIG. 2, respectively.

As shown in FIG. 4, the sleeve-shaped connector 13A is coaxially fixed to the ring-shaped fixing member 27, which fastens the piezoelectric tubular actuator 16 to the tip unit 10B. Flexible wiring boards 29 composed of any suitable material, such as polyimide film, extend on the outer surface between the fixing member 27 and the end portion of the sleeve-shaped connector 13A. One end portion of the flexible wiring boards 29 is attached to the end portion of the connector 13A to connect with the receptacles 21A to 21D shown in FIG. 2, whereas the other end portion of the flexible wiring boards 29 is attached to the piezoelectric actuator 16 and the photodiodes 14. Thus, the piezoelectric actuator 16 is electrically connected to the signal cable CB2 shown in FIG. 1 via the flexible wiring boards 29.

The plurality of photodiodes 14 (not shown in FIG. 4) are arranged around the actuator 16, and are fixed to the fixing member 27 circumferentially, at given constant intervals. The photodiodes 14 are connected to the flexible board 29 so that the photo-diodes 14 are electrically connected to the signal cable CB1 via the flexible wiring boards 29.

The vibrating optical fiber 17 projects from the actuator 16 along the axis L so that the distal end of the optical fiber 17 becomes a cantilever. An adhesive 26 may be applied to the end portion of the actuator 16 to hold the cantilevered vibrating optical fiber 17 securely. The tube-shaped piezoelectric actuator 16 may be a bimorph type, and may be formed of any suitable piezoelectric materials, such as PZT. The piezoelectric actuator 16 deforms by the inverse piezoelectric effect, and two-dimensionally drives the distal end of the vibrating optical fiber 17. The piezoelectric actuator 16 vibrates the distal end along two axes perpendicular to each other while modulating or amplifying amplitudes of the vibration, so as to scan the tip portion of the vibrating optical fiber 17 in spiral patterns. Thus, light irradiated from the tip unit 10B via the lens 19 is scanned over the observed portion in the spiral patterns.

Light reflected from the observed portion passes through the lens 19 and the plurality of photodiodes 14 collect the reflected light. In this manner, signals corresponding to the detected light are read from the photodiodes 14 in a time-series, and are fed to the image signal processing circuit 44 shown in FIG. 1. R, G, and B color filters may be disposed, respectively, on the photodiodes 14 such that the balance or ratio of the colors R, G, and B are usually equal. In the image signal processing circuit 44, color in each pixel is detected from signals fed from the plurality of photodiodes 14. For example, when the ratio of signals fed from photodiodes with an R filter is larger than that fed from photo-diodes with the other (G, B) filters, the pixel color is set to a reddish color.

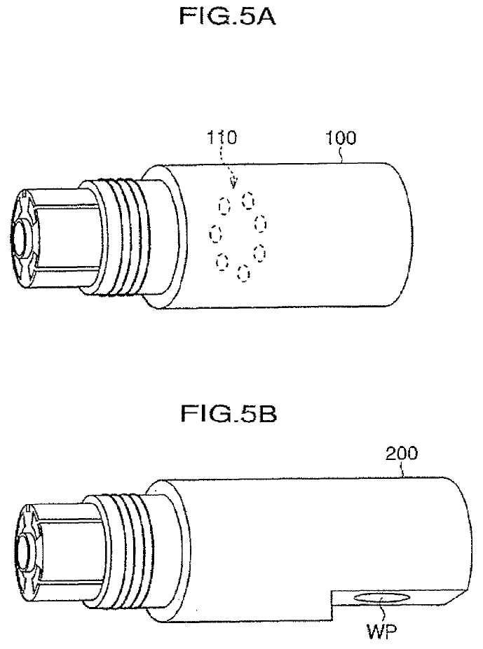

FIGS. 5A and 5B show different types of tip units, which have structures similar to the tip unit 10B shown in FIGS. 1 to 3, as discussed above.

FIG. 5A shows a tip unit 100 which is an auto-fluorescent observation type tip unit, which has a plurality of filters 110. Each filer 110 is disposed in front of a corresponding photo-detector, and blocks light having an ultraviolet wavelength (so called “excitation-light”). The other structures of the tip unit 100 are similar to those of the forward observation type tip unit 10B depicted in FIGS. 2-4. When the excitation-light is emitted from the tip unit 100 via an optical fiber, the excitation-light is reflected off the tissue, and fluorescent light emitted from an observed tissue is radiated to the tip unit 100. The excitation-light is blocked by the filter 110 so that only the auto-fluorescent light reaches the photo-diodes.

FIG. 5B shows a tip unit 200 which is a side observation type tip unit, in which a window WP is disposed opposite the side direction with respect to an axis, namely, the longitudinal direction of the tip unit 200. In the tip unit 200, a mirror or prism for reflecting light is provided. The other structures of the tip unit 200 are similar to those of the forward observation type tip unit 10B depicted in FIGS. 2-4.

In this manner in the first embodiment, the tip unit 10B equipped with the optical fiber 17, the photodiodes 14, and the actuator 16 is interchangeable and detachably connected to the distal end 10C of the insertion portion 10A. When the tip unit 10B is attached to the insert portion 10A, the actuator 16 and the photodiodes 14 are electrically connected to the signal cables CB1 and CB2 via the flexible wiring boards 29. Also, the optical fiber 17 is optically connected to the optical fiber 12.

An operator may select a proper tip unit from the three types of tip units 10B, 100, and 200, and attach a selected tip unit to the distal end of the insertion portion 10A. The operator can then change the observation condition by changing only the tip unit, without changing an entire endoscope body. Further, the tip units can be disposable or single-use, to avoid additional cleaning and sterilization steps. Further still, since the optical fiber 17 is disposed along the axis L of the housing 11 of the endoscope tip unit 10B, the connector 13A can be formed as a sleeve, which is a relatively simple construction.

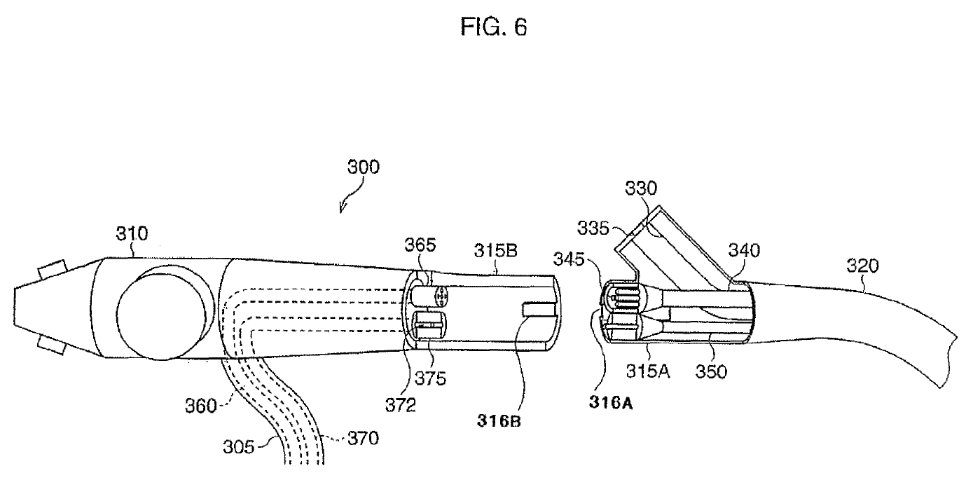

With reference to FIG. 6, a second embodiment of an endoscope system is explained. The second embodiment is different from the first embodiment in that an insertion portion is detachably attached to an endoscope. Otherwise, the construction is generally the same as that of the first embodiment.

In an endoscope 300, a tube 305 extends from a processor to an operation portion 310. The operation portion 310 includes operator controls, such as knobs or buttons, for manipulating or bending the distal end of the endoscope 300. A tubular and flexible insertion portion 320 with a connector 315A is attached to the operation portion 310 via a connector 315B. A forceps tube or channel 330 is provided in the insertion portion 320. An instrument may be inserted through an inlet 335 of the forceps channel 330. The insertion portion 320 is a forward observation type. In the distal end of the insertion portion 320, an optical fiber, a piezoelectric actuator and photodiodes are provided, similar to the first embodiment.

A signal cable 340 extends through the insertion portion 320, and a plurality of pins 345 are attached to the end portion of the signal cable 340. A signal cable 360, which extends through the tube 305 and the operation portion 310, is connected to a socket or receptacle 365 provided in the connector 315B.

An optical fiber 370 extending through the tube 305 and the operation portion 310 is connected to a ferrule 372, which is coaxially inserted in a sleeve 375 provided in the connector 315B. The connector 315A fits in the connector 315B, so that the signal cable 340 is electrically connected to the signal cable 360 via the receptacle 365 in which pins 345 are received. The fiber cable 370 is optically connected to the fiber cable 350 via the ferrule 372. The connectors 315A and 315B may include respective alignment structures, such as a groove 316A and a corresponding protrusion 316B, in order to ensure proper alignment during connection.

In the second embodiment, the insertion portion 320 is removably or detachably connected to the operation portion 310. In this manner, the insertion portion 320 is interchangeable and may be a of single-use type. For example, another type of insertion portion, such as an auto-fluorescent observation type or a side-observation type, can be attached to the operation portion 310.

In the first and second embodiments, although a tip unit with photodiodes is shown, other types of photo-detectors may be utilized. Further, a tip unit with an image-guide composed of an optical fiber, which optically transmits an image from the distal end to the proximal end of the endoscope, can also be used. In addition, a tip unit of co-focal construction with double cladding may be utilized.

Finally, it will be understood by those skilled in the arts that the foregoing description is of preferred embodiments of the device, and that various changes and modifications may be made to the present invention without departing from the spirit and scope thereof.

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The above disclosed subject matter is to be considered illustrative, and not restrictive, and the appended claims are intended to cover all such modifications, enhancements, and other embodiments which fail within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

Although the invention has been described with reference to several exemplary embodiments, it is understood that the words that have been used are words of description and illustration, rather than words of limitation. As the present invention may be embodied in several forms without departing from the spirit or essential characteristics thereof it should also be understood that the above-described embodiments are not limited by any of the details of the foregoing description, unless otherwise specified. Rather, the above-described embodiments should be construed broadly within the spirit and scope of the present invention as defined in the appended claims. Therefore, changes may be made within the metes and bounds of the appended claims, as presently stated and as amended, without departing from the scope and spirit of the invention in its aspects.

What is claimed is:

1. An endoscope comprising:
an insertion portion configured to be inserted into an object, said insertion portion having a signal cable and a first optical fiber extending therethrough; and
a tip unit removably attached to a distal end of said insertion portion, said tip unit including a second optical fiber optically connected to said first optical fiber, and an actuator electrically connected to said signal cable, said actuator vibrating said second optical fiber so as to scan light irradiated from said second optical fiber over an observed area.

2. The endoscope according to claim 1, wherein said tip unit further comprises a flexible board that electrically connects said actuator with said signal cable.

3. The endoscope according to claim 2, wherein said flexible board is arranged laterally with respect to said second optical fiber.

4. The endoscope according to claim 1, further comprising:
a pin electrically connected to said signal cable and provided at the distal end of said insertion portion; and
a receptacle provided at a connecting portion of said tip unit and configured to receive said pin.

5. The endoscope according to claim 1, wherein said tip unit further comprises a ferrule that connects said second optical fiber with said first optical fiber.

6. The endoscope according to claim 5, further comprising a sleeve that accommodates said ferrule.

7. The endoscope according to claim 1, wherein said second optical fiber is a single mode optical fiber.

8. The endoscope according to claim 1, wherein said actuator comprises a piezoelectric actuator.

9. The endoscope according to claim 1, wherein said actuator vibrates a tip portion of said second optical fiber in spiral patterns.

10. The endoscope according to claim 1, wherein said tip unit further comprises at least one optical lens located at a tip portion of said tip unit.

11. The endoscope according to claim 1, wherein said tip unit further comprises at least one photodetector electrically connected to said signal cable and configured to convert light reflected or emitted by the observed area to image-pixel signals.

12. The endoscope according to claim 1, wherein said tip unit further comprises at least one filter that blocks part of light reflected or emitted by the observed area.

13. The endoscope according to claim 1, wherein said tip unit supports said second optical fiber along a longitudinal axis of said endoscope tip unit.

14. The endoscope according to claim 1, wherein said tip unit is configured to detect auto-fluorescence emitted by the observed area.

15. The endoscope according to claim 1, wherein said tip unit is configured for side viewing of the observed area.

16. An endoscope tip unit comprising:
a connector that has an electrical connection portion and an optical connection portion;
an optical fiber that is optically connected to said optical connection portion;
an actuator that is electrically connected to said electrical connection portion, said actuator vibrating said optical fiber so as to scan light irradiated from said optical fiber over an observed area; and
a tubular housing that accommodates said optical fiber,
wherein said tip unit is configured to be removably attached to a distal end of an endoscope insertion portion.

17. The endoscope tip unit according to claim 16, wherein said optical fiber is disposed along a longitudinal axis of said endoscope tip unit.

18. The endoscope tip unit according to claim 16, wherein said connector comprises a sleeve that is coaxial to said housing.

19. The endoscope tip unit according to claim 16, wherein said optical connection portion is coaxial to said housing.

20. The endoscope tip unit according to claim 19, wherein said optical connection portion comprises a ferrule that optically connects said optical fiber with said optical connection portion.

21. The endoscope tip unit according to claim 20, wherein said connector comprises a sleeve that coaxially accommodates said ferrule.

22. The endoscope tip unit according to claim 16, further comprising a flexible board that electrically connects said actuator with said electrical connection portion.

23. The endoscope tip unit according to claim 16, wherein said actuator comprises a tubular piezoelectric actuator, said optical fiber being passed through said actuator along a longitudinal axis of said endoscope tip unit.

24. The endoscope tip unit according to claim 16, wherein said actuator vibrates a tip portion of said optical fiber in spiral patterns.

25. The endoscope tip unit according to claim 16, further comprising at least one optical lens located at a tip portion of said endoscope tip unit.

26. The endoscope tip unit according to claim 16, further comprising at least one photodetector electrically connected to said electrical connection portion and configured to convert light reflected or emitted by the observed area to image-pixel signals.

27. The endoscope tip unit according to claim 16, further comprising at least one filter that blocks a part of light reflected or emitted by the observed area.

28. The endoscope tip unit according to claim 16, wherein said electrical connection portion has a receptacle.

29. The endoscope tip unit according to claim 16, wherein said optical fiber is a single mode optical fiber.

30. The endoscope tip unit according to claim 16, wherein said tip unit is configured to detect auto-fluorescence emitted by the observed area.

31. The endoscope tip unit according to claim 16, wherein said tip unit is configured for side viewing of the observed area.

32. The endoscope tip unit according to claim 16, wherein said electrical connection portion is configured to connect to a signal cable of the endoscope insertion portion.

33. The endoscope tip unit according to claim 16, wherein said optical connection portion is configured to connect to an optical fiber of the endoscope insertion portion.

34. An endoscope comprising:
an operation portion that is operated to manipulate a distal end of said endoscope, a first signal cable and a first optical fiber extending from a proximal end of said endoscope through said operation portion; and
a tubular insertion portion configured to be inserted into an object and removably attached to said operation portion,
wherein said insertion portion includes a second optical fiber optically connected to said first optical fiber, a second signal cable electrically connected to said first signal cable, and an actuator electrically connected to said second signal cable and vibrating a tip portion of said second optical fiber so as to scan light irradiated from said second optical fiber over an observed area.

35. The endoscope according to claim 34, wherein said insertion portion further includes an instrument channel.
